(19) United States
(12) Patent Application Publication
(10) Pub. No.: US 2020/0170827 A1
(43) Pub. Date: Jun. 4, 2020

(54) Distributive Foot Sleeve for Relieving Pressure in a Ski Boots and the Like

(71) Applicant: Arched Bow LLC, Memphis, TN (US)

(72) Inventor: Bradley L. Ottinger, Memphis, TN (US)

(73) Assignee: Arched Bow LLC, Memphis, TN (US)

(21) Appl. No.: 16/616,458

(22) PCT Filed: May 23, 2018
(86) PCT No.: PCT/US2018/034154

§ 371 Date: Nov. 23, 2019

Related U.S. Application Data
(60) Provisional application No. 62/510,305, filed on May 24, 2017.

(51) Int. Cl. A61F 5/34; A43B 5/04

(52) U.S. Cl. CPC A61F 5/34; A43B 5/0405

(57) ABSTRACT

A distributive foot sleeve for relieving pressure in the foot and ankle area of a user when inserted into footgear, such as a ski boot. One or more compartments in the foot sleeve are configured to distribute pressure from areas of high pressure to areas of lower pressure during an activity, such as via a gel material in the one or more compartments. The gel material may be formulated to remain viscous at temperatures between about 10 to about 100 degrees Fahrenheit so as to maintain pressure distribution properties in a wide range of operating conditions for footwear.

### CROSS REFERENCE TO RELATED APPLICATIONS

This application claims the benefit of: PCT/US2018/034154 (WO2018/217908A1) filed May 23, 2018; and U.S. Provisional Patent Application 62/510,305 filed May 24, 2017. The foregoing references are incorporated herein by reference in their entirety.

### FIELD OF THE INVENTION

The present invention relates to protective footgear, and more particularly to specialized footgear for relieving discomfort and pressure in the area of the foot and ankle.

### BACKGROUND OF THE INVENTION

Ski boots are widely regarded as uncomfortable, due to the need for rigid and tight control of the lower leg while skiing. Individuals who ski frequently typically invest in customized ski boots. The cost of customized boots is high, and is impractical for individuals who ski infrequently.

Individuals who suffer from conditions of the foot or ankle, such as arthritis, frequently experience increased pain when engaged in physically demanding activities such as skiing, hiking, running, or manual labor. While various efforts have been made to provide specialized footgear to relive pain and discomfort, a need remains for an economical, off-the-shelf solution that can be turned to on an as-needed basis, such as on a once a year ski trip.

### OBJECTS AND SUMMARY OF THE INVENTION

It is an object of the inventions to provide a specialized footgear for relieving pressure around the foot and ankle when enclosed in footwear, such as a ski boot.

It is an object of the inventions to provide removable and replaceable footgear for relieving pressure around the foot and ankle when enclosed in footwear.

The foregoing objectives are achieved by providing a distributive foot sleeve having the features described herein.

The distributive foot sleeve comprises a sleeve body. The sleeve body includes a foot portion, an ankle portion extending from the foot portion, an open proximal end on the ankle portion, and a distal end on the foot portion. A sleeve wall extends between the proximal and distal ends. The sleeve wall has an exterior side and an interior side. An internal bore extends along the interior side between the proximal and distal ends for closely receiving the foot and ankle region of a user therein. The sleeve body is configured to distribute and relieve pressure when pressurized between the foot of the user and the interior of the footwear. The configuration for distributing and relieving pressure comprises the sleeve body formed of a multilayered structure comprising an interior layer along the interior side, an exterior layer along the exterior side, and a viscous gel layer sandwiched and enclosed within the interior and exterior layers. The sleeve body is separate from the footwear.

The gel layer may comprise a gel material formulated to remain viscous at temperatures between about 10 to about 100 degrees Fahrenheit so as to maintain pressure distribution properties in a wide range of operating conditions for footwear. The gel material may be enclosed in an impenetrable but malleable enclosure in order to contain the gel material and allow the gel material to readily move from high pressure to low pressure areas when pressurized. The gel material may be compartmentalized in two or more individual compartments within the sleeve, whereby forces are distributed and equalized within the two or more individual compartments. The two or more individual compartments may include a compartment in the foot portion and a compartment in the ankle portion.

The exterior layer may comprise a slippery or low friction material to facilitate ease of insertion and removal of the sleeve from the footwear. The slippery or low friction material of the exterior layer may be selected from the group consisting of silk, rayon, nylon, polyester, or a blend thereof. The interior layer may comprise a soft material for comfort of the user, such as neoprene.

The sleeve body may further comprise a collar portion extending upward from the ankle portion to receive a shin or lower leg of the user. The distal area of the foot portion may be open to allow toes of the user to pass therethrough, or the distal area may be enclosed in the manner of a sock. The ankle portion may include opposing medial and lateral ankle openings sized and positioned to accommodate protrusions of medial and lateral malleolus bones of the user. A rear of the foot portion may have an opening positioned to accommodate a calcaneus of the user.

The distributive foot sleeve may be provided as a pair of foot sleeves having the foregoing characteristics to thereby provide a pair of foot sleeves for use with a pair of the footwear. In some embodiments, the foot sleeve is configured to equalize pressure in a ski boot.

Methods of distributing and relieving pressure in a foot and ankle area of a foot of a user in an interior of a footwear are provided, including providing a foot sleeve device having the foregoing characteristics, inserting the foot of the user into the foot sleeve, inserting the foot sleeve and inserted foot into the footwear, and using the foot sleeve and footwear combination for an activity. During use in the activity, pressure between an interior of the footwear and the foot of the user equalizes and distributes pressure in the gel layer to provide cushion, support and comfort to the user. Providing may include providing a pair of foot sleeve devices for use with a pair of footwear. The footwear may be a ski boot, with the foot sleeve device configured to equalize pressure in a ski boot.

The foregoing and other objects, features, aspects and advantages of the invention will become more apparent from the following detailed description of the invention when considered in conjunction with the accompanying drawings.

### BRIEF DESCRIPTION OF THE DRAWINGS

FIG. 1 is a front perspective view of one embodiment of the invention.

FIG. 2 is a side perspective view of one embodiment of the invention.

FIG. 3 is a front cross-section view showing one embodiment of the invention around the foot and ankle of a user.

FIG. 4 is front break-away view showing one embodiment of layering of an interior wall of the invention.

FIG. 5 is a cross-section view showing layers used in one embodiment of the invention.

FIG. 6 is a side view showing a representative view of a pressure relieving cavity or compartment.

### PREFERRED EMBODIMENTS OF THE INVENTION

In the following detailed description of the preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the present invention.

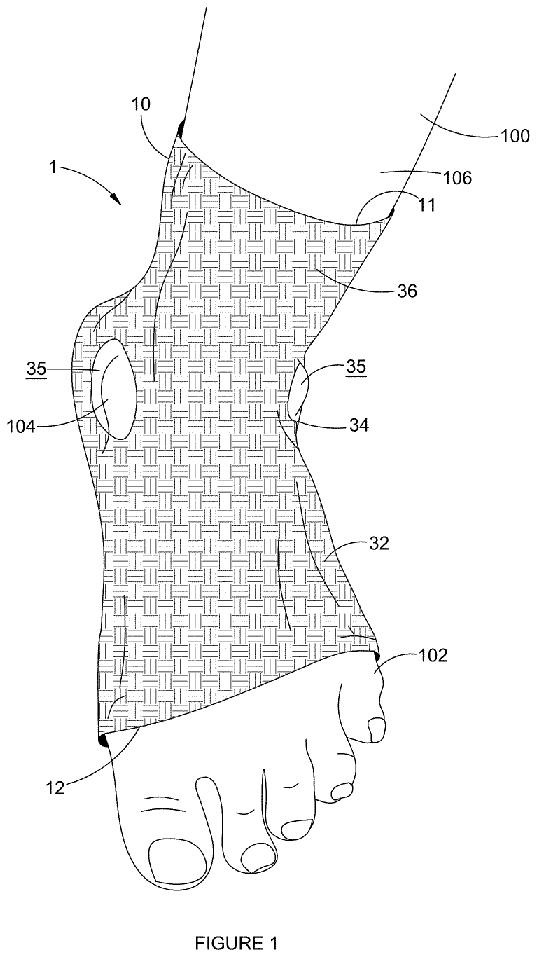

As shown in FIG. 1, the invention 1 comprises, generally, a sleeve body 10 configured to receive a foot of a user 100 therein. The sleeve body 10 includes an open proximal end 11, an open distal end 12, and a sleeve wall 14 extending between the proximal and distal ends 11, 12. The wall 14 has an exterior side 16 and interior side 17. An internal bore 20 extends along the interior side between the proximal and distal ends 11, 12 for closely receiving a foot 102 and ankle 104 region of a user 100 therein during use.

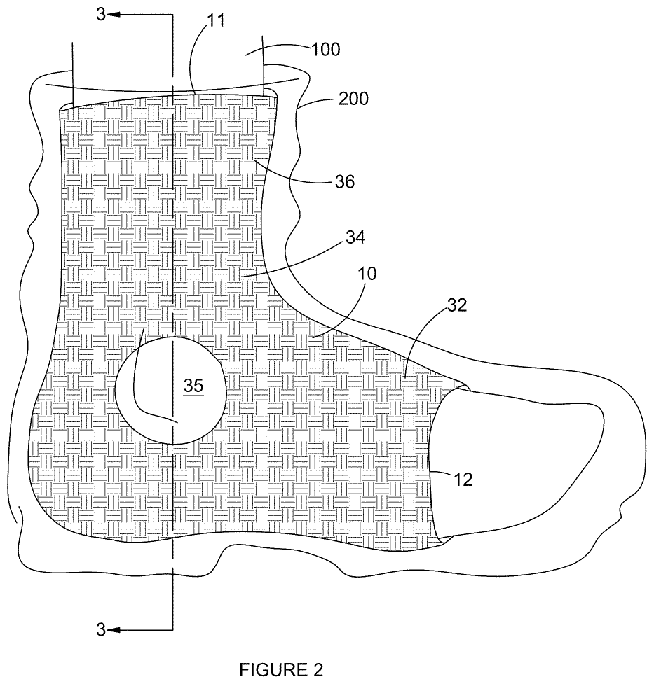

As shown in FIG. 2, the sleeve body 10 includes a foot portion 32 for receiving a foot 102 of a user 100, a collar portion 36 extending upward to receive a shin or lower leg 106 of a user 100, and an ankle portion 34 disposed between the foot portion 32 and collar portion 36 for receiving an ankle 104 of a user 100. In the embodiment of FIG. 2, the ankle portion 34 includes opposing medial and lateral ankle openings 35 for accommodating the protrusions of the medial and lateral ankle bones (anatomically known as the medial and lateral malleolus) of a user 100.

An opening can also optionally be provided along the area of the heal (calcaneus), particularly in situations where bulk in the heal area may not be desirable or required. In some embodiments, the toe area of the sleeve body 10 may be fully enclosed such that distal end 12 is closed in the manner of a sock. Opposing medial and lateral ankle openings 35 can be eliminated such that the ankle portion 34 is fully enclosed. The collar portion 36 can be eliminated for cosmetic reasons or for low demand uses, such as in leisure or dress shoes.

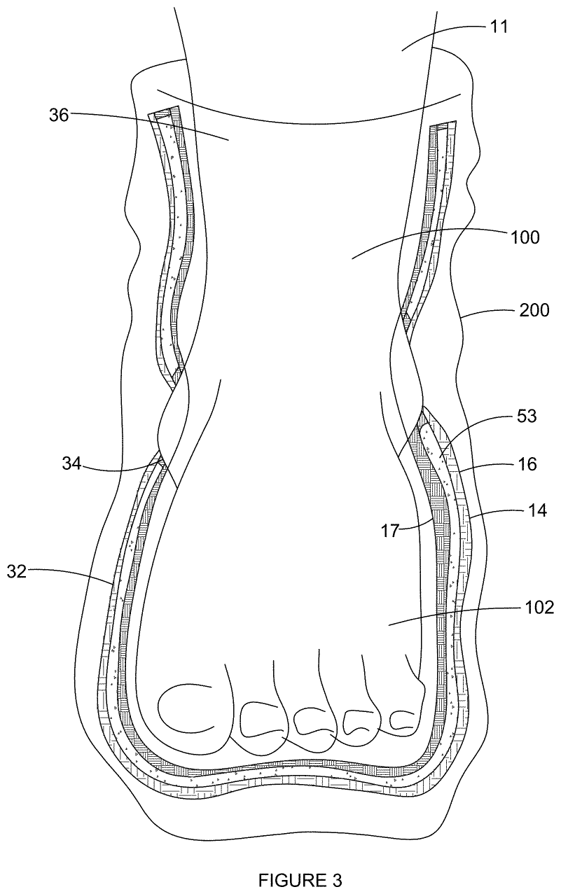

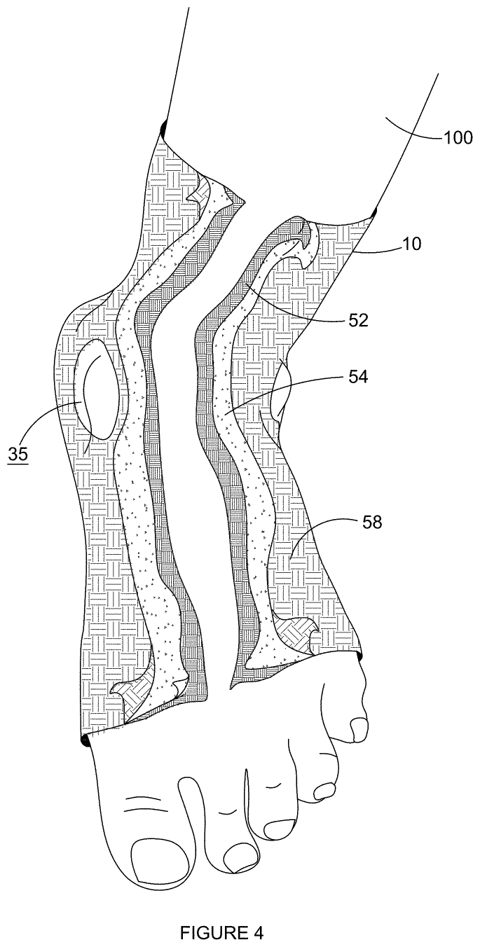

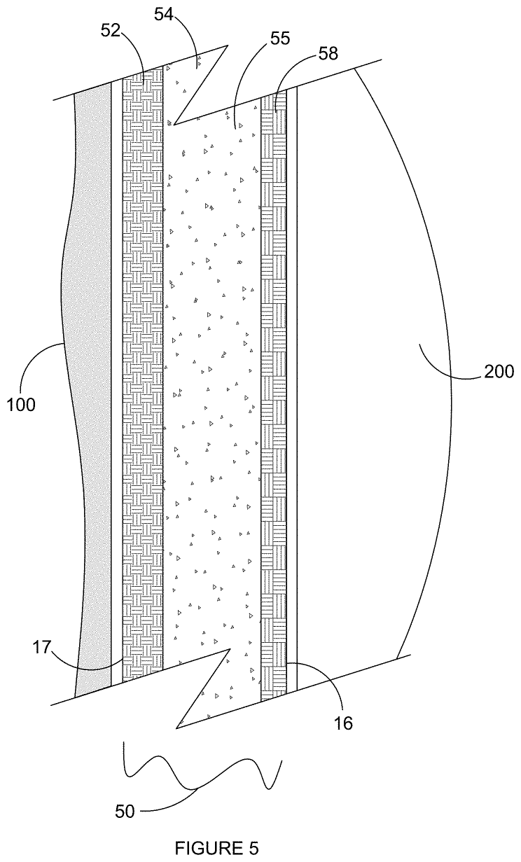

Force distributive features of the distributive foot sleeve 1 of the invention will now be described. As shown in FIGS. 3-5, the sleeve body 10 is formed from a multilayered structure 50. The multilayered structure 50 includes an interior layer 52 comprising a soft material, an exterior layer 58 comprising a slippery or low friction outer surface, and a viscous gel 54 sandwiched and enclosed within the interior and exterior layers 52, 58.

In an exemplary embodiment, the interior layer 52 is a thin neoprene. Neoprene provides elasticity to hold the sleeve 1 in place on the user 100, strength to contain the gel and to support the foot and ankle of the user 100, softness to avoid skin abrasion, compressibility to assist with comfort and load distribution, and insulation for warmth such as when skiing or hiking in cold weather.

In an exemplary embodiment, the outer layer 58 has a slippery or low friction exterior surface to reduce friction and aid in boot entry and exit. In non-limiting examples, the outer layer 58 may be made of silk, rayon, nylon, polyester, or a blend of such materials.

In an exemplary embodiment, the gel layer 54 comprises a gel material 55. The gel is formulated to remain viscous at temperatures normally encountered during use, such as 10 to 100 degrees Fahrenheit. The gel material 55 may be enclosed in an impenetrable but malleable enclosure or compartment 53, such as plastic or rubber, in order to contain the gel 54 and allow the gel 54 to readily move from high pressure to low pressure areas around the foot 102 of the user 100.

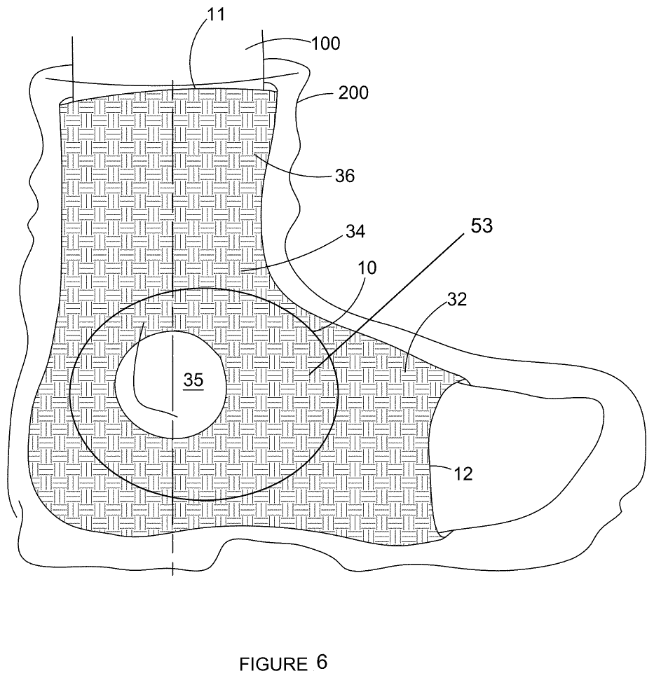

As indicated in FIGS. 3 and 6, the viscose gel 54 may be compartmentalized in one or more compartments 53 within the sleeve 10, such as a compartment enclosing the foot, a compartment enclosing the ankle, and a compartment enclosing the shin. In this manner, forces are distributed and equalized within the individual compartments. Various physical activities will require more or less amounts of pressure distribution in various areas of the foot, which can be accomplished through the use of one or more compartments 53. FIG. 6 provides a representative view of a compartment 53 positioned in the ankle area. Compartments 53 could be provided on both sides of the ankle bone area, as well as on other areas, such as a compartment 53 on top of the foot, a pair of compartments 53 on opposing sides of the shin area, or various combinations thereof. While the compartment 53 is shown as being oval, the compartment can take various forms to meet the needs of the activity or the space available in the footwear, such as round, square, rectangular, triangular, polygonal, and various combinations thereof

The foot sleeve 10 of the invention 1 is intended to provide a standard off-the-shelf but customized foot gel compartment 53. The foot sleeve 10 will be provided in various sizes to allow use by a wide range of potential users. The foot sleeve 10 may be provided in specific foot sizes using conventional foot size designations, such as size 8, 9, 10 and so forth. The foot sleeve 10 may further be provided in various widths designated with convention foot width designations, such as width C, D and E. However, since it is an object of the invention to provide a generally economical solution that can be readily discarded and replaced as-needed, a subset of sizes may be selected to fit a large portions of the user population, such as small, medium, large, extra-large, and the like.

While the invention 1 has been described primarily with reference to ski boots, it will be appreciated that the invention can be used in any footwear in which relief of pressure around the food and ankle is desired. Non-limiting applications of the invention 1 include hiking boots, work boots, athletic shoes, dress shoes, and leisure shoes.

In use, the invention 1 is worn on the foot of a user in the manner of a conventional sock. However, when the sleeve body 10 is inserted into the interior of a ski boot or other footwear, the gel 54 evenly distributes forces within the cavity between the interior surface of the footwear and the foot 102 and ankle 104 of the user. The configuration of the distributive foot sleeve 1 allows distribution of forces to take place naturally between the interior of the footwear 200 and the exterior of the user's foot, ankle and lower leg 102, 104, 106. Compression between the footwear 200 and the user's anatomy causes the viscose gel material 54 to matriculate from areas of high pressure to areas of low pressure to fully encapsulate the lower leg, foot and ankle and distribute pressure evenly across surface areas of the leg, ankle and foot. By equally distributing pressure in this manner, high pressure areas (the shin, the ankle bones, the arch and the top of the foot) are spared undue pressure, circulation is improved, pain decreased and overall comfort improved without sacrificing stability and control.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. A device for relieving pressure in a foot and ankle region of a user when inserted into an interior of a footwear, comprising:
a sleeve body, the sleeve body including
a foot portion,
an ankle portion extending from the foot portion,
an open proximal end on the ankle portion,
a distal end on the foot portion,
a sleeve wall extending between the proximal and distal ends, the sleeve wall having an exterior side and interior side,
an internal bore extending along the interior side between the proximal and distal ends for closely receiving a foot and ankle region of said user therein,
the sleeve body configured to distribute and relieve pressure when pressurized between said foot of said user and said interior of said footwear, the configuration for distributing and relieving pressure comprising the sleeve body formed of a multilayered structure comprising an interior layer along the interior side, an exterior layer along the exterior side, and a viscous gel layer sandwiched and enclosed within the interior and exterior layers, and
the sleeve body being separate from said footwear.

2. The device of claim 1, wherein the gel layer comprises a gel material formulated to remain viscous at temperatures between about 10 to about 100 degrees Fahrenheit so as to maintain pressure distribution properties in a wide range of operating conditions for footwear.

3. The device of claim 2, wherein the gel material is enclosed in an impenetrable but malleable enclosure in order to contain the gel material and allow the gel material to readily move from high pressure to low pressure areas when pressurized.

4. The device of claim 2, wherein the gel material is compartmentalized in two or more individual compartments within the sleeve, whereby forces are distributed and equalized within the two or more individual compartments.

5. The device of claim 4, wherein the two or more individual compartments include a compartment in the foot portion and a compartment in the ankle portion.

6. The device of claim 1, wherein the exterior layer comprises a slippery or low friction material to facilitate ease of insertion and removal of the sleeve from said footwear.

7. The device of claim 6, wherein the slippery or low friction material of the exterior layer is selected from the group consisting of silk, rayon, nylon, polyester, or a blend thereof.

8. The device of claim 2, wherein the interior layer comprises a soft material for comfort of said user.

9. The device of claim 8, wherein the soft material of the interior layer comprises neoprene.

10. The device of claim 1, the sleeve body further comprising a collar portion extending upward from the ankle portion to receive a shin or lower leg of said user.

11. The device of claim 1, wherein the distal area of the foot portion is open to allow toes of said user to pass therethrough.

12. The device of claim 1, wherein the ankle portion includes opposing medial and lateral ankle openings sized and positioned to accommodate protrusions of medial and lateral malleolus bones of said user.

13. The device of claim 1, wherein a rear of the foot portion has an opening positioned to accommodate a calcaneus of said user.

14. The device of claim 1, wherein a toe area of the foot portion is fully enclosed such that the distal end is closed in the manner of a sock.

15. The device of claim 1, further comprising a second foot sleeve according to claim 1 to thereby provide a pair of foot sleeves for use with a pair of said footwear.

16. The device of claim 1, wherein the foot sleeve is configured to equalize pressure in a ski boot.

17. A method of distributing and relieving pressure in a foot and ankle area of a foot of a user in an interior of a footwear, comprising
providing a foot sleeve device according to claim 1,
inserting said foot of said user into the foot sleeve,
inserting the foot sleeve and inserted foot into said footwear,
using the foot sleeve and footwear combination for an activity, whereby during use in the activity, pressure between an interior of said footwear and said foot of said user equalizes and distributes pressure in the gel layer to provide cushion, support and comfort to the user.

18. The method of claim 17, wherein providing comprises providing a pair of foot sleeve devices for use with a pair of footwear.

19. The method of claim 17, wherein the footwear is a ski boot and the foot sleeve device is configured to equalize pressure in a ski boot.
